(19) United States
(12) Patent Application Publication
(10) Pub. No.: US 2026/0193703 A1
(43) Pub. Date: Jul. 9, 2026

(54) HIGH-THROUGHPUT AMPLIFICATION OF TARGETED NUCLEIC ACID SEQUENCES

(71) Applicant: BIOSEARCH TECHNOLOGIES, INC., PETALUMA, CA (US)

(72) Inventors: ERIC STEINMETZ, MIDDLETON, WI (US); MICHAEL LODES, MIDDLETON, WI (US); TONY ROCKWEILER, MIDDLETON, WI (US); SCOTT MONSMA, MIDDLETON, WI (US); DIEGO FAJARDO, MIDDLETON, WI (US); JASON WALKER, MIDDLETON, WI (US); DIPANKAR MANNA, MIDDLETON, WI (US)

(21) Appl. No.: 19/131,606

(22) PCT Filed: Nov. 21, 2023
(86) PCT No.: PCT/US2023/080693

§ 371 Date: May 21, 2025

Related U.S. Application Data
(60) Provisional application No. 63/426,913, filed on Nov. 21, 2022.

(51) Int. Cl. C12Q 1/6874; C12Q 1/6806

(52) U.S. Cl. CPC C12Q 1/6874; C12Q 1/6806

(57) ABSTRACT

The disclosure pertains to materials and methods for capturing a target nucleic acid sequence, comprising annealing a first target specific oligonucleotide primer to a target sequence; elongating the 3′ end of the first target specific oligonucleotide primer to linearly amplify the target nucleic acid sequence. Then, annealing a second target specific oligonucleotide primer to the amplified target sequence; elongating the 3′ end of the second target specific oligonucleotide primer to linearly amplify the complement to the target nucleic acid sequence. The resulting copies of the target nucleic acid sequence can be detected or sequenced. A plurality of target nucleic acid sequences from one or more samples can also be captured. Unique identifier sequences can be introduced to track the source of the captured target nucleic acid sequence. The invention also provides kits for performing the methods disclosed herein.

### CROSS-REFERENCE TO RELATED APPLICATION

This application claims the benefit of U.S. Provisional Application Ser. No. 63/426,913, filed Nov. 21, 2022, the disclosure of which is hereby incorporated by reference in its entirety, including all figures and tables.

### BACKGROUND OF THE INVENTION

Targeted sequencing is growing in importance as more robust and affordable sequencing technologies become available. The majority of the conventional methods for analyzing target nucleic acid sequences involve target hybridization and capture (Gnirke et al., 2009), multiplex PCR (Campbell et al., 2015) or molecular inversion probes (Shen et al., 2011). These methods are either expensive, difficult to optimize, have high data variability, or lack flexibility to sequence targets of different length. Therefore, improved methods are desirable for analyzing, such as detecting and sequencing, target nucleic acid sequences.

### BRIEF SUMMARY OF THE INVENTION

Certain embodiments disclosed herein provide materials and methods for amplifying target nucleic acid sequences and/or genomic regions and optionally, further analyzing the target sequences, such as by detection and/or sequencing.

In certain embodiments, the methods disclosed herein for amplifying a target sequence comprise combining a first target specific oligonucleotide primer and a DNA polymerase, wherein the target specific oligonucleotide primer comprises at least 10 nucleotides that are complementary to the nucleic acid sequence of interest and a first adaptor sequence (which can also be referred to as a “Read 1” sequence in the examples) that is non-complementary to the sequence of interest. The first adaptor sequence can optionally comprise a restriction enzyme recognition site. The first target specific oligonucleotide primer and target sequence can then be amplified by the DNA polymerase, thus linearly amplifying the target nucleic acid sequence. In certain embodiments, the products of the amplification reaction can be digested with a restriction enzyme specific to the restriction enzyme recognition site in the first adaptor sequence, eliminating primer-dimers.

In certain embodiments, the products of the amplification reaction or restriction enzyme digestion can be diluted by the addition of a second target specific oligonucleotide primer and DNA polymerase, wherein the second target specific oligonucleotide primer comprises a portion with at least 10 bases that are complementary to the amplified nucleic acid sequence of interest and a second adaptor sequence (which can also be referred to as a “Read 2” sequence in the examples) non-complementary to the sequence of interest.

The second target specific oligonucleotide primer and the amplified target sequence can then be amplified by a DNA polymerase and the second target specific oligonucleotide primer, thus providing a nucleic acid sequence complementary to the amplified target sequence.

In certain embodiments, the amplified target sequence nucleic acid and the sequence complementary to the amplified target sequence can be combined with a first tagging oligonucleotide primer (for example, a first indexing primer) that anneals to the complement of the first adaptor sequence and a second tagging oligonucleotide primer for example, a second indexing primer) that anneals to a complement of the second adaptor sequence to amplify the nucleic acid sequences of interest, resulting in a library of tagged sequences of interest when amplified.

In certain embodiments, the library of tagged sequences of interest are suitable for further detection and/or sequencing. Sequencing can be performed using next generation sequencing techniques such as, nanopore sequencing, reversible dye-terminator sequencing, Single Molecule Real-Time (SMRT) sequencing or paired-end sequencing.

In certain embodiments, a plurality of target sequences in a sample are captured using a plurality of first target specific oligonucleotide primers and, in a subsequent amplification reaction, a plurality of second target specific oligonucleotide primers and a plurality of first and second tagging primers, amplifying the second target specific oligonucleotide primers annealed to the corresponding target sequences (or complements thereof) to capture the plurality of target sequences. Oligonucleotide primers can further be used to produce double-stranded copies of the target sequences that are suitable for further detection and sequencing. A plurality of first and second tagging primers can be combined with a plurality of amplified target nucleic acid samples to sequence in a multiplex sequencing reaction. The first and second tagging primers can comprise unique identifier sequences to identify the source of the amplified target sequences. After the sequencing step, the sample specific unique identifiers are used to allocate a sequence to a sample and the sequence of the captured target sequences. Sequencing can be performed using next generation sequencing techniques such as, nanopore sequencing, reversible dye-terminator sequencing, Single Molecule Real-Time (SMRT) sequencing, or paired-end sequencing.

Further embodiments of the invention provide kits for carrying out the methods disclosed herein. The kits comprise one or more of: one or more pairs of target specific oligonucleotide primers and one or more pairs of tagging primers, enzymes, such as DNA polymerase, reagents for sequencing, and instructions for conducting the assays.

### BRIEF DESCRIPTION OF THE DRAWINGS

FIG. 1. Overview of one example of the two-stage process of annealing a “forward” primer and amplifying a target nucleotide sequence. Following the initial amplification, a “reverse” primer is combined with the amplified product from the first amplification reaction along with indexing primers for amplifying and then sequencing target nucleic acid sequences, according to the methods disclosed herein.

FIGS. 2A-2B. Bioanalyzer fractionated samples to evaluate the library profiles.

FIG. 3. The sequencing results of two pools of libraries combined for sequencing on an Illumina MiSeq platform.

FIG. 4. A comparison of the proportion of targets called consistently in all 4 replicates, or only in 1, 2, or 3 of the 4 replicates.

FIG. 5. A comparison of the proportion of uncalled targets in single replicates, or in combinations of 2, 3 or 4 replicates.

FIG. 6 presents a schematic illustration of anticipated products of a first linear amplification reaction performed with only Adaptor 1-containing Forward primers, including the intended single-stranded extension products and some potential double-stranded products arising from primer-dimer interactions or off-target priming.

FIGS. 7A-7B show bioanalyzer traces of libraries produced with a panel of 960 primer pairs targeting regions of interest within the soy genome. FIG. 7A is an example of products of library preparation following the Linear/Exponential protocol, in which products of the first linear amplification reaction performed with Forward primers only were utilized directly in a second exponential amplification with Reverse primers and indexing primers without restriction enzyme treatment. Products include a major peak of primer-dimer sized products as well as a broad distribution of products of apparent sizes up to 10 kb. A minority of products are consistent with expected library fragment sizes. FIG. 7B shows products from the same primer pools and protocol, except that Stage 1 products were treated with restriction enzyme BspQI (New England Biolabs) before initiation of Stage 2 cycling. The major products are library fragments of the expected size (~300-450 bp) and a small amount of primer-dimer sized products (150-170 bp).

FIGS. 8A-8E show bioanalyzer traces for libraries prepared from HotSHOT extracts without dilution, or from extracts that had been diluted with an equal volume of either 40 mM Tris-HCl at a pH of 5.0 or water. Control libraries were produced with purified Maize B73 DNA (10 ng) or no DNA.

FIG. 9 presents key metrics from the sequence analysis of the high-quality libraries produced from HotSHOT crude extract samples with the Linear/Exponential method, with >99% of reads mapped to target loci for all 3 conditions. Genotype calls were made for 97% to 98% of target loci at an average sequencing depth of 139 reads per target, and very high Uniformity of target coverage (88-90%) was achieved.

### DETAILED DISCLOSURE OF THE INVENTION

As used herein, the singular forms “a”, “an” and “the” are intended to include the plural forms as well, unless the context clearly indicates otherwise. To the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrase “consisting essentially of” or “consists essentially of” indicates that the described embodiment encompasses embodiments containing the specified materials or steps and those that do not materially affect the basic and novel characteristic(s) of the described embodiment.

The term “about” means within an acceptable error range for the particular value as determined by one of ordinary skill in the art, which will depend in part on how the value is measured or determined, i.e., the limitations of the measurement system. In the context of the lengths of polynucleotides where the terms “about” are used, these polynucleotides contain the stated number of bases or base-pairs with a variation of 0-10% around the value (X±10%).

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, such as for the size of the polynucleotides, the combinations and sub-combinations of the ranges (e.g., subranges within the disclosed range) and specific embodiments therein, are explicitly included.

The term “organism” as used herein includes viruses, bacteria, fungi, plants and animals. Additional examples of organisms are known to a person of ordinary skill in the art and such embodiments are within the purview of the materials and methods disclosed herein. The assays described herein can be useful in analyzing any genetic material obtained from any organism. In certain embodiments, the organism can be an animal, such as, for example, a fruit fly, nematode worm, fish, human, mouse, rat, dog, cat, horse, frog, sheep, cow, donkey, goat, deer, llama, pig, chicken, alpaca, rabbit, or guinea pig. In certain embodiments, the organism is a plant, such as, for example, Arabidopsis thaliana, maize/corn, legume, tobacco, or rice.

The term “genome”, “genomic”, “genetic material” or other grammatical variation thereof as used herein refers to genetic material from any organism. A genetic material can be viral genomic DNA or RNA, nuclear genetic material, such as genomic DNA, or genetic material present in cell organelles, such as mitochondrial DNA or chloroplast DNA. It can also represent the genetic material coming from a natural or artificial mixture or a mixture of genetic material from several organisms.

As used herein, “a target genomic region” is a region of interest in a genetic material of an organism. As used herein, “a target sequence”, “target nucleic acid sequence”, “a sequence of interest”, or “a target sequence of interest” is a region of interest in a synthetic nucleic acid sequence, plasmid, or genetic material of an organism, microbiome, or virus. These terms can be used interchangeably within this application. In certain embodiments, the genetic material can be derived from a bacteriophage or an environmental microbiome.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

As used herein, an “isolated” or “purified” nucleic acid molecule or polynucleotide is substantially free of other compounds, such as cellular material, with which it is associated in nature. A purified or isolated polynucleotide (ribonucleic acid (RNA) or deoxyribonucleic acid (DNA)) can be free of the genes or sequences that flank it in its naturally-occurring state. Alternatively, an “isolated” or “purified” nucleic acid molecule or polynucleotide may be RNA or genomic DNA purified from its naturally occurring source, such as a prokaryotic or eukaryotic cell and/or cellular material with which it is associated in nature.

As used herein, a “crude” nucleic acid or polynucleotide sample contains other compounds, such as cellular material, with which it is associated in nature. A crude polynucleotide (ribonucleic acid (RNA) or deoxyribonucleic acid (DNA)) sample contains genes or sequences that flank it in its naturally-occurring state. Non-limiting examples include prokaryotic and eukaryotic cell lysates.

The term “hybridizes with” or “anneals to” when used with respect to two sequences indicates that the two sequences are sufficiently complementary to each other to allow nucleotide base pairing between the two sequences. Sequences that hybridize or anneal with each other can be perfectly complementary but can also have mismatches to a certain extent. Therefore, the sequences at the 5′ and 3′ ends of the primers described herein may have a few mismatches with the corresponding target sequences at the 5′ and 3′ ends of the target nucleotide sequences as long as the primers can hybridize with the target sequences to facilitate capturing of the target nucleotide sequence. Depending upon the stringency of hybridization, a mismatch of up to about 5% to 20% between the two complementary sequences would allow for hybridization between the two sequences. Typically, high stringency conditions have higher temperature and lower salt concentration and low stringency conditions have lower temperature and higher salt concentration. High stringency conditions for hybridization are preferred, and therefore, the sequences at the 3′ and 5′ ends of the primers are preferred to be perfectly complementary to the corresponding target sequences at the 3′ and 5′ ends of the target nucleic acid sequence.

The term “identifier”, “tag”, or “tagging sequence” as used herein refers to a known nucleotide sequence of between four to one hundred nucleotides, preferably, between ten to twenty nucleotides, and even more preferably, about eight or sixteen nucleotides. The appropriate length of tag sequences depends on the sequencing technology being used. Once incorporated into the amplified target nucleotide sequences, the tagging sequences can facilitate sequencing and identification of the target nucleotide sequences, for example, by providing unique identification sites that allow allocating the correct sequences to the correct target nucleotide sequences.

The term “paired-end sequencing” used herein refers to the sequencing technology where both ends of a double-stranded polynucleotide are sequenced using specific primer binding sites present on each end of the double-stranded polynucleotide. Paired-end sequencing generates high-quality sequencing data, which is aligned using a computer software program to generate the sequence of the polynucleotide flanked by the two primer binding sites. Sequencing from both ends of a double-stranded molecule allows high quality data from both ends of the double-stranded molecule because sequencing from only one end of the molecule may cause the sequencing quality to deteriorate as longer sequencing reads are performed.

In the paired-end sequencing, the double-stranded amplified target sequences produced at the end of the final PCR amplification step of the methods disclosed herein are sequenced using specific primers that bind to the two ends of the double-stranded target sequences. A general description and the principle of paired-end sequencing is provided in Illumina Sequencing Technology, Illumina, Publication No. 770-2007-002, the contents of which are herein incorporated by reference in their entirety.

Non-limiting examples of the paired-end sequencing technology are provided by Illumina MiSeq™, Illumina MiSeqDx™ and Illumina MiSeqFGx™. Additional examples of the paired-end sequencing technology that can be used in the assays disclosed herein are known in the art and such embodiments are within the purview of the invention.

As used herein, the phrase “hairpin adapter” refers to a polynucleotide containing a double-stranded stem and a single-stranded hairpin loop. The single-stranded hairpin loop region of a hairpin adapter can provide primer binding site for sequencing. Thus, once a hairpin adapter hybridizes with both sticky ends of a target nucleic acid sequences, it produces a double-stranded DNA template containing the target nucleic acid sequences in the double-stranded region capped by hairpin loops at both ends. Such template can be used for sequencing the target nucleic acid sequences via Single Molecule Real-Time (SMRT) sequencing (PacBio™). Description and the principle of SMRT sequencing is provided in Pacific Biosciences (2018), Publication No.: BR108-100318, the contents of which are herein incorporated by reference in their entirety.

Nanopore technology may be used in the methods disclosed herein to sequence the target nucleic acid sequences. In certain such embodiments, the copies of target nucleic acid sequences are processed to sequence the target nucleic acid sequences as described, for example, in Nanopore Technology Brochure, Oxford Nanopore Technologies (2019), and Nanopore Product Brochure, Oxford Nanopore Technologies (2018). The contents of both these brochures are herein incorporated by reference in their entireties.

Throughout this disclosure, different sequences are described by specific nomenclature, for example, forward primer sequence, reverse primer sequence, and tagging primer sequence. When such nomenclature is used, it is understood that the identified sequence is substantially identical or substantially reverse complementary to at least a part of the corresponding sequence. For example, “a primer sequence” describes a sequence that is substantially identical to at least a part of the primer sequence or substantially reverse complementary to at least a part of the primer sequence. This is because when a captured target nucleic acid sequence is converted into a double-stranded form comprising the primer binding sequence, the double-stranded target nucleic acid sequence can be sequenced using a primer having a sequence that substantially identical or substantially reverse complementary to at least a part of primer binding sequence. Thus, the nomenclature is used herein to simplify the description of different polynucleotides and parts of polynucleotides used in the methods disclosed here; however, a person of ordinary skill in the art would recognize that appropriate substantially identical or substantially reverse complementary sequences to at least a part of the corresponding sequences could be used to practice the methods disclosed herein.

Also, two sequences that correspond to each other, for example, a primer sequence or a tagging primer sequence, have at least 90% sequence identity, preferably, at least 95% sequence identity, even more preferably, at least 97% sequence identify, and most preferably, at least 99% sequence identity, over at least 70%, preferably, at least 80%, even more preferably, at least 90%, and most preferably, at least 95% of the sequences. Alternatively, two sequences that correspond to each other are reverse complementary to each other and have at least 90% perfect matches, preferably, at least 95% perfect matches, even more preferably, at least 97% perfect matches, and most preferably, at least 99% perfect matches in the reverse complementary sequences, over at least 70%, preferably, at least 80%, even more preferably, at least 90%, and most preferably, at least 95% of the sequences. Thus, two sequences that correspond to each other can hybridize with each other or hybridize with a common reference sequence over at least 70%, preferably, at least 80%, even more preferably, at least 90%, and most preferably, at least 95% of the sequences. Preferably, two sequences that correspond to each other are 100% identical over the entire length of the two sequences or 100% reverse complementary over the entire length of the two sequences.

This disclosure provides materials and methods that solve the problems associated with conventional methods for analyzing target nucleic acid sequences. Particularly, this disclosure provides materials and methods for analyzing a target nucleic acid sequence. In certain embodiments, the target nucleic acid sequence can be purified. Alternatively, the sample containing target nucleic acid can be in a crude form. In certain embodiments, a cell lysing agent can be added to a crude sample. For example, DNA or RNA can be purified from a mixture by extraction with a solvent or resin, precipitation, electrophoresis, chromatography, or a combination thereof. Alternatively, the RNA or DNA may be used with no or a minimum of purification to avoid losses due to sample processing. The RNA or DNA may be dried for storage or dissolved in an aqueous solution. The solution may contain buffers or salts to promote annealing, and/or stabilization of the duplex strands.

In certain embodiments, for amplification of nucleic acids from crude extracts, certain additives may be included in the amplification reaction. In certain embodiments, the additives can include, for example, bovine serum albumin (BSA); single-stranded DNA binding protein (SSB); dimethylsulfoxide (DMSO); nonionic detergents, such as, for example Tween-20 or ectoine; or any combination thereof.

In certain embodiments, the detection of the at least one single-stranded or double-stranded nucleic acid is carried out in an enzyme-based nucleic acid amplification method. The expression “enzyme-based nucleic acid amplification method” relates to any method wherein enzyme-catalyzed nucleic acid synthesis occurs. Such an enzyme-based nucleic acid amplification method can be preferentially selected from the group constituted of LCR, Q-beta replication, NASBA, LLA (Linked Linear Amplification), TMA, 3SR, Polymerase Chain Reaction (PCR), notably encompassing all PCR based methods known in the art, such as reverse transcriptase PCR (RT-PCR), simplex and multiplex PCR, real time PCR, end-point PCR, quantitative or qualitative PCR and combinations thereof. These enzyme-based nucleic acid amplification method are well known to the man skilled in the art and are notably described in Saiki et al. (1988) Science 239:487, EP 200 362 and EP 201 184 (PCR); Fahy et al. (1991) PCR Meth. Appl. 1:25-33 (3SR, Self-Sustained Sequence Replication); EP 329 822 (NASBA, Nucleic Acid Sequence-Based Amplification); U.S. Pat. No. 5,399,491 (TMA, Transcription Mediated Amplification), Walker et al. (1992) Proc. Natl. Acad. Sci. USA 89:392-396 (SDA, Strand Displacement Amplification); EP 0 320 308 (LCR, Ligase Chain Reaction); Bustin & Mueller (2005) Clin. Sci. (London) 109:365-379 (real-time Reverse-Transcription PCR). In some embodiments, the enzyme-based nucleic acid amplification method is selected from the group consisting of Polymerase Chain Reaction (PCR) and Reverse-Transcriptase-PCR (RT-PCR).

In certain embodiments, the target nucleic acid sequence can be RNA or DNA. RNA or DNA can be artificially synthesized or isolated from natural sources. In some embodiments, the RNA target nucleic acid sequence can be a ribonucleic acid such as RNA, mRNA, piRNA, tRNA, IRNA, ncRNA, gRNA, shRNA, siRNA, snRNA, miRNA and snoRNA More preferably the DNA or RNA is biologically active or encodes a biologically active polypeptide. The DNA or RNA template can also be present in any useful amount.

Reverse transcriptases useful in the present invention can be any polymerase that exhibits reverse transcriptase activity. Preferred enzymes include those that exhibit reduced RNase H activity. Several reverse transcriptases are known in the art and are commercially available (e.g., from Biosearch Technologies, Middleton, WI; Bio-Rad Laboratories, Inc., Hercules, CA; Boehringer Mannheim Corp., Indianapolis, Ind.; Life Technologies, Inc., Rockville, Md.; New England Biolabs, Inc., Beverley, Mass.; Perkin Elmer Corp., Norwalk, Conn.; Pharmacia LKB Biotechnology, Inc., Piscataway, N.J.; Qiagen, Inc., Valencia, Calif.; Stratagene, La Jolla, Calif.). In some embodiments, the reverse transcriptase can be Avian Myeloblastosis Virus reverse transcriptase (AMV-RT), Moloney Murine Leukemia Virus reverse transcriptase (M-MLV-RT), Human Immunovirus reverse transcriptase (HIV-RT), EIAV-RT, RAV2-RT, C. hydrogenoformans DNA Polymerase, rTth DNA polymerase, SUPERSCRIPT I, SUPERSCRIPT II, and mutants, variants and derivatives thereof. It is to be understood that a variety of reverse transcriptases can be used in the present invention, including reverse transcriptases not specifically disclosed above, without departing from the scope or preferred embodiments disclosed herein.

DNA polymerases useful in the present invention can be any polymerase capable of replicating a DNA molecule. Preferred DNA polymerases are thermostable polymerases and polymerases that have exonuclease activity, which are especially useful in PCR. Thermostable polymerases are isolated from a wide variety of thermophilic bacteria, such as Thermus aquaticus (Taq), Thermus brockianus (Tbr), Thermus flavus (Tfl), Thermus ruber (Tru), Thermus thermophilus (Tth), Thermococcus litoralis (Tli) and other species of the Thermococcus genus, Thermoplasma acidophilum (Tac), Thermotoga neapolitana (Tne), Thermotoga maritima (Tma), and other species of the Thermotoga genus, Pyrococcus furiosus (Pfu), Pyrococcus woesei (Pwo) and other species of the Pyrococcus genus, Bacillus sterothemophilus (Bst), Sulfolobus acidocaldarius (Sac) Sulfolobus solfataricus (Sso), Pyrodictium occultum (Poc), Pyrodictium abyssi (Pab), and Methanobacterium thermoautotrophicum (Mth), and mutants, variants or derivatives thereof.

Many DNA polymerases are known in the art and are commercially available (e.g., Biosearch Technologies, Middleton, WI; from Bio-Rad Laboratories, Inc., Hercules, CA; Boehringer Mannheim Corp., Indianapolis, Ind.; Life Technologies, Inc., Rockville, Md; New England Biolabs, Inc., Beverley, Mass.; Perkin Elmer Corp., Norwalk, Conn.; Pharmacia LKB Biotechnology, Inc., Piscataway, N.J.; Qiagen, Inc., Valencia, Calif.; Stratagene, La Jolla, Calif.). In some embodiments, the DNA polymerase can be Taq, Tbr, Tfl, Tru, Tth, Tli, Tac, Tne, Tma, Tih, Tfi, Pfu, Pwo, Kod, Bst, Sac, Sso, Poc, Pab, Mth, Pho, ES4, VENT™, DEEPVENT™, and active mutants, variants and derivatives thereof. It is to be understood that a variety of DNA polymerases can be used in the present invention, including DNA polymerases not specifically disclosed above, without departing from the scope or preferred embodiments thereof.

In certain embodiments, the target sequence can be obtained from a sample from, for example, an environmental sample, including, for example, a water sample, an air sample, a surface or equipment sample, or a soil sample. An additional example is the environment on a farm, slaughterhouse or any other location where food is processed (e.g., packing houses). Samples from a farm would include soil samples, surfaces on farm buildings, farm equipment. Recreational water is any water in which recreation occurs and includes recreational bodies of water such as swimming pools, lakes, rivers, oceans, etc. In certain embodiments, the water or soil sample may contain a microbiome. Surfaces are relevant particularly in hospitals, schools, or food processing facilities. In certain embodiments, the food processing samples can comprise samples from meat, fish, plants, or fungi to determine to genetical material present in the sample. The samples can be swabs taken from surfaces and the swab is then introduced into the medium from which droplets are created. In some embodiments, the sample is a sample from a subject (e.g., a human subject) to determine a genetic sequence present in the subject, or the subject may be known or suspected of having genetic abnormalities or of being infected by a pathogenic microorganism or virus. The sample can be blood, or a fraction thereof such as plasma or serum; tissue, urine, saliva; pericardial, pleural or spinal fluids; sputum, bone marrow stem cell concentrate, platelet concentrate; nasal, rectal, vaginal or inguinal swabs; wounds; specimens from skin, mouth, tongue, throat; ascites; stools and the like. The disclosed methods can also be used to identify target nucleic acid sequences within the microbiota of a subject from sources such as soil microbiomes, gastrointestinal microbiomes, vaginal microbiomes, skin microbiomes, oral microbiomes, and/or respiratory microbiomes.

The methods disclosed herein provide capturing a target nucleic acid sequence. The methods comprise the steps of:
a) annealing a first target specific oligonucleotide primer to a target sequence, wherein: the first target specific oligonucleotide primer comprises a first target binding sequence toward a 3′ end and a first adaptor sequence toward a 5′ end;
b) amplifying the target nucleic acid sequence by extending the 3′ end of the first target specific oligonucleotide primer;
c) adding a second target specific oligonucleotide primer, a first tagging primer, and a second tagging primer to the amplified target nucleic acid sequence, wherein:
the second target specific oligonucleotide primer comprises a second target binding sequence complementary to the amplified target nucleic acid sequence toward a 3′ end and a second adaptor sequence toward a 5′ end, and
the first tagging primer anneals to a complement of the first adaptor sequence and the second tagging primer anneals to a complement of the second adaptor sequence; and
d) amplifying the complementary sequence to the amplified target nucleic acid sequence by extending the 3′ end of the second target specific oligonucleotide primer and amplifying the target sequence and complement thereof using the first and second tagging primers, yielding a library of tagged target sequences. Steps a) and b), above, can be repeated one or more times prior to the performance of steps c) and d).

The methods disclosed herein also provide capturing a target nucleic acid sequence. The methods comprise the steps of:
a) annealing a first target specific oligonucleotide primer to a target sequence, wherein:
the first target specific oligonucleotide primer comprises a first target binding sequence toward a 3′ end and a first adaptor sequence toward a 5′ end;
b) amplifying the target nucleic acid sequence by extending the 3′ end of the first target specific oligonucleotide primer;
c) repeating steps a) and b) at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 75, 80, 85, 90, 95, or 100 times;
d) adding a second target specific oligonucleotide primer, a first tagging primer, and a second tagging primer to the amplified target nucleic acid sequence, wherein:
the second target specific oligonucleotide primer comprises a second target binding sequence complementary to the amplified target nucleic acid sequence toward a 3′ end and a second adaptor sequence toward a 5′ end, and
the first tagging primer anneals to a complement of the first adaptor sequence and the second tagging primer anneals to a complement of the second adaptor sequence; and
e) amplifying the complementary sequence to the amplified target nucleic acid sequence by extending the 3′ end of the second target specific oligonucleotide primer and amplifying the target sequence and complement thereof using the first and second tagging primers, yielding a library of tagged target sequences.

The first target specific oligonucleotide primer comprises toward the 3′ end a sequence that anneals with a first target sequence. Such sequence on the first target specific oligonucleotide primer is referenced herein as the first target binding sequence. The first target specific oligonucleotide primer comprises toward the 5′ end a first adaptor sequence that is preferably non-complementary to the first target sequence, i.e., the adaptor sequence has less than about 70%, about 60%, about 50%, about 40%, about 30%, about 20%, about 10%, or 0% sequence identity to the nucleic acid sequence of interest. The first target binding sequence and the first adaptor sequence may have an intervening or otherwise additional sequence that can provide additional functionality, such as, an identifier sequence.

The second target specific oligonucleotide primer comprises toward the 3′ end a sequence that anneals with a second target sequence. Such sequence on the second target specific oligonucleotide primer is referenced herein as the second target binding sequence. The second target specific oligonucleotide primer comprises toward the 5′ end a second adaptor sequence that is preferably non-complementary to the second target sequence, i.e., the adaptor sequence has less than about 70%, about 60%, about 50%, about 40%, about 30%, about 20%, about 10%, or 0% sequence identity to the nucleic acid sequence of interest. The second target binding sequence and the second adaptor sequence may have an intervening sequence that can provide additional functionality, such as, an identifier sequence.

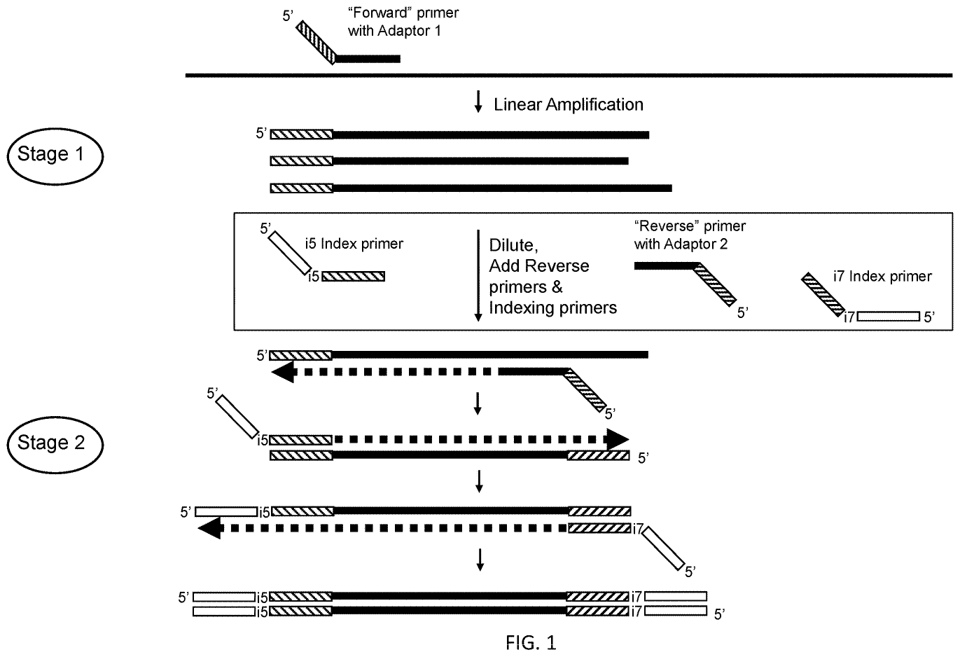

Thus, the methods disclosed herein comprise two distinct steps of annealing of a first specifically designed oligonucleotide primer to a certain target sequence and amplifying the certain target sequence and a second distinct step of annealing a second specifically designed oligonucleotide primer to the amplification products of the previous step. FIG. 1 shows a target nucleic acid sequence. The first “forward” oligonucleotide primer (shown on the left in Step 1 of FIG. 1) is referenced herein as “the forward primer” and the second “reverse” oligonucleotide primer (shown on the right in Step 2 of FIG. 1) is referenced herein as “the reverse primer”. The sequence at the 3′ end of the forward primer anneals to the corresponding target sequence and the sequence at the 3′ end of the reverse primer anneals to the corresponding target sequence towards the 3′ end of the linearly amplified target sequence that results from the first amplification reaction.

Each of the forward and reverse primer can contain a minimum of between about 20 and about 60 nucleotides. The first target binding sequence of the forward primer can be at least between about 10 and about 30 nucleotides. Similarly, the second target binding sequence of the reverse primer can be at least between about 10 and about 30 nucleotides. The specificity of the primer towards the target binding sites can be controlled by the lengths of the first and the second target sequences. Particularly, longer lengths of the first and the second target sequences provide higher binding specificity and shorter lengths of the first and the second target sequences provide lower specificity. A person of ordinary skill in the art can determine appropriate sequences for the first and the second target sequences based on the sequence of the target nucleic acid sequence and the available sequences for a particular organisms, plasmids, or viruses, for example, from a genome sequence database.

In certain embodiments, at least one oligonucleotide primer useful in the provided methods can incorporate nucleic acid modifications that can enhance or alter the performance of the oligonucleotide primer. In certain embodiments, at least one phosphorothioate modification can be incorporated in the oligonucleotide primer to stabilize the oligonucleotide primer against digestion by proof-reading polymerases with 3′-5′ exonuclease activity. In certain embodiments, alternative backbone chemistries, such as, for example, locked nucleic acid (LNA) or peptide nucleic acid (PNA), can be incorporated in the oligonucleotide primer, which can enhance sensitivity or specificity of primer-template interactions. In certain embodiments, at least one modified base, such as, for example, deoxyuridine, can be incorporated in the oligonucleotide primer, which can provide a mechanism for the degradation of the oligonucleotide primer through treatment with a combination of Uracil deglycosylase and Endonuclease VIII.

The length of the target nucleic acid sequence and, hence, the distance between target sequences of the two primers depends on the purpose of the analysis, the characteristics of the target nucleic acid sequence, and when performed, the sequencing methods used for the analysis. For example, if Illumina™ 2×150 bp sequencing method is used, target sequences of about 300 bp are analyzed. If paired-end or nanopore based sequencing technique is used, target sequences of about 1,000 bp to about 20,000 bp can be analyzed.

In the methods disclosed herein, the target sequences comprise about 10 bp and about 100 bp, between about 100 bp and about 300 bp, between about 300 bp and about 1,000 bp, between about 1,000 bp and about 20,000 bp, preferably, about 2 bp to about 500 bp, more preferably, about 100 bp to about 500 bp, or, most preferably, about 300 to about 500 bp. Therefore, the two primers hybridize non-adjacently on the target nucleic acid sequences.

At the end of the first annealing step, the forward primer is annealed to the first target sequence via the first target binding sequence and the target nucleic acid sequence is amplified. In the second annealing step, the reverse primer is annealed to the second target sequence via the second target binding sequence. The first and the second target binding sequences can flank the target nucleic acid sequence or the first and second target binding sequence can be a portion of the target nucleic acid sequence.

The methods disclosed herein further comprise an elongation reaction to elongate the forward primer, i.e., to extend the forward primer. The elongation of the forward primer is designed to amplify the target nucleic acid sequence. The methods disclosed herein further comprise an elongation reaction to elongate the reverse primer, i.e., to extend the reverse primer. The elongation of the reverse primer is designed produce an amplified sequence that is complementary to the target nucleic acid sequence. The extension of the of the forward and reverse primers can be carried out using a DNA polymerase.

In preferred embodiments, no purification step is used after one or more amplification step within the disclosed methods. In certain embodiments of the methods disclosed herein, one or more of the amplification steps can be followed by a step designed to remove from the reaction mixture unwanted material, such as unincorporated primers, extension products, for example, and the target nucleic acid sequence. Such a step is optional.

In preferred embodiments, after an amplification step (e.g., after step b)), the amplification products are diluted with the addition of, for example, a buffer, one or more primers (e.g., a target specific oligonucleotide primer, a tagging primer), polymerase, metal ions, deoxyribonucleotides (dNTPs), restriction enzyme, water, or any combination thereof. In certain embodiments, the amplification product is diluted by a factor of about 5× to about 100×, about 5× to about 50×, about 5× to about 30×, or, preferably, about 5×.

Peng et al., 2015 (Peng Q, Vijaya Satya R, Lewis M, Randad P, Wang Y., Reducing amplification artifacts in high multiplex amplicon sequencing by using molecular barcodes, BMC Genomics, 2015 Aug. 7; 16(1): 589, doi: 10.1186/s12864-015-1806-8. PMID: 26248467; PMCID: PMC4528782) presents a method in which a first linear amplification reaction incorporating 1 to 3 rounds of thermal cycling is performed with a tagged and barcoded first primer pool. Then, after extensive and required purification, a PCR is performed with a second tagged primer pool in combination with a universal primer complementary to the tag on the first primer pool (see, for example, page 4 column 1: “After evaluating several approaches, we found that two-round size selection purification is the most efficient way to remove primer dimer background (Additional file 1: Figure S1)” and page 11 column 2: “Figure S1: Two rounds of size selection purification efficiently removed unused BC primers and as a result eliminated any primer dimer problem.”). The authors further state that, “[t]he keys to success in high multiplex amplicon barcoding PCR are minimizing primer dimer formation . . . ”. Further, the Peng et al. reference does not disclose the use of a restriction enzyme digestion to remove unwanted material after an amplification reaction, superficially primer-dimers. In Peng et al., after a second purification, a pair of universal primers is used to complete the addition of adapter sequences through PCR. The indexing primer sequences in the subject method contain universal portions as well as unique index (barcode) sequences.

In the subject methods, the step b) reaction products, including enriched target sequences, are diluted before the step d) reaction containing the second target specific oligonucleotide primer pool and the inclusion of two tagging primers in this second-stage reaction to enable finished library construction without intermediate purification steps. The subject methods do not require purification after the first linear amplification (step b)); instead, the first-stage reaction is diluted (step c)) with the components required for the second-stage reaction (step d)). The second-stage reaction includes the second target specific oligonucleotide primer pool, along with two indexing primers containing complementarity to the first and second target specific oligonucleotide primer pools. The subject methods do not require purification prior to the final amplification by the indexing primers. Thus, in some embodiments, the methods disclosed herein can be performed without purification of intermediate amplification products (such as that required in Peng et al.).

In certain embodiments, the removal of unwanted material is performed using a restriction enzyme, particularly primer-dimers that are formed during the amplification process. The restriction enzyme can have activities towards single-stranded and, preferably, double-stranded nucleic acids. Non limiting examples of exonucleases that can be used in the methods disclosed herein include Type I, Type II, Type III, Type IV, and Type V. A suitable restriction enzyme and recognition site can be selected by a person of ordinary skill in the art.

In certain embodiments, unintended off-target products produced by primers combining to amplify regions other than their intended targets may be removed from the library by treatment with oligonucleotide-directed nucleases, such as, for example, CRISPR-Cas or argonaute enzymes.

In certain embodiments, a pair of tagging primers can be added simultaneously to the reverse primer or after the addition of the reverse primer (shown in Step 2 of FIG. 1). In certain embodiments, the first tagging primer anneals to the complement of the first adaptor sequence and the second tagging primer anneals to a complement of the second adaptor sequence to amplify the nucleic acid sequence interest, resulting in a library of tagged target sequences. The use of the tagging primers is designed to serve any one or a combination of purposes, the amplification of the target sequences, for example, via PCR, to detectable levels; the incorporation of sample-specific identifiers (also referenced in the art as indexes, barcodes, zip codes, adapters, etc.), and the incorporation of sequences that facilitate sequencing of the target nucleic acid sequences.

In certain embodiments the tagging primer pair comprises a first tagging primer that comprises a sequence that anneals to the complement of the first adaptor sequence, i.e., identical or sufficiently identical to the first adaptor sequence and a second tagging primer that comprises a sequence that anneals to the complement of the second adaptor sequence, i.e., identical or sufficiently identical to the second adaptor sequence. In this step, a PCR is used to amplify the nucleic acid sequence of interest using a tagging primer pair.

The tagging primer pair can be designed so that the resulting double-stranded amplified target sequence, in addition to the first and second target binding sequences, further comprises one or more of: a first sequencing primer binding sequence, a first identifier sequence, a second sequencing primer binding sequence and a second identifier sequence.

In certain embodiments, one or both primers of the tagging primer pair comprise additional sequences that can facilitate downstream sequencing of the double-stranded target nucleic acid sequences produced at the end of the amplification step. The additional sequences that can facilitate sequencing can contain, for example, at least a portion of the sequences required for flow-cell binding and sequencing primer binding to initiate sequencing on Illumina™ platform, such as paired-end or single-read sequencing, at least a portion of the hair-pin adapter required for hairpin adapter based sequencing, such as PacBio sequencing, or at least a portion of the sequences required for properly guiding the molecules through a nanopore technology based sequencer. When the resulting molecule contains only a portion of the sequences required for sequencing, the remainder can be introduced by any other fashion know in the art, such as adapter ligation.

In any of the amplification steps, in addition to primers and target nucleic acid sequence, the PCR reaction mixture may contain a DNA polymerase and other reagents for PCR, such as dNTPs, metal ions (for example, Mg²⁺ and Mn²⁺), and a buffer. In certain preferred embodiments, the master mix containing RapiDxFire Hot Start Taq DNA Polymerase (Biosearch Technologies, Hoddesdon, UK) is used in the subject methods. Additional reagents which may be used in a PCR reaction are well-known to a person of ordinary skill in the art and such embodiments are within the purview of the invention.

Typically, a PCR comprises about 5 to about 40 cycles or about 25 to about 40 cycles, each cycle comprising a step of denaturation, annealing, and extension at different temperatures. A step of final extension can be performed at the end of the last cycle of the PCR. Designing various aspects of a PCR, including the number of cycles and durations and temperatures of various steps within the cycle is apparent to a person of ordinary skill in the art and such embodiments are within the purview of the invention.

When the forward primer anneals with the target nucleic acid sequence, the structure provided in FIG. 1, step 1, is produced. Thus, during the initial cycles of the PCR, the complementary copies of the target sequence are produced with all components of the forward primer. In the second cycle of the PCR, the reverse primer anneals to the amplified target nucleic acid sequences and amplifies a nucleic acid sequence complementary to the initial amplified target nucleic acid sequence and the tagging primers bind the adaptor regions of the forward and reverse primers in FIG. 1, step 2, yielding double-stranded copies of the target nucleic acid sequence.

At the end of the PCR cycling, multiple copies of the target nucleic acid sequences are produced that are suitable for further analysis, such as detection or sequencing. In certain embodiments, the tagging primers can comprise a sequencing/indexing primer binding sequence, (e.g., a sequence that can be recognized by an i5 or i7 indexing primer). An example of such double-stranded DNA is provided in FIG. 1, step 3. This double-stranded DNA comprises from one end to the other, the sequences corresponding to one or more of: an i5 indexing sequence, first adaptor sequence, first target sequence, a target nucleic acid sequence, second target sequence, second adaptor sequence, i7 indexing sequence, and any additional sequences that can facilitate sequencing of the double-stranded DNA containing the target nucleic acid sequence.

The amplified target sequence can be detected using techniques known in the art, for example, using a labeled probe complementary to a sequence within the target sequence. For example, the amplified target sequence can be detected based on the turbidity of the reaction, fluorescence detection or labeled molecular beacons.

The term “label” refers to a molecule detectable by spectroscopic, photochemical, biochemical, immunochemical, chemical, or other physical means. For example, useful labels include fluorescent dyes (fluorophores), fluorescent quenchers, luminescent agents, electron-dense reagents, biotin, digoxigenin, ³²P and other isotopes or other molecules that can be made detectable, e.g., by incorporating into an oligonucleotide. The term includes combinations of labeling agents, e.g., a combination of fluorophores each providing a unique detectable signature, e.g., at a particular wavelength or combination of wavelengths.

Exemplary fluorophores include, but are not limited to, Alexa dyes (e.g., Alexa 350, Alexa 430, Alexa 488, etc.), AMCA, BODIPY 630/650, BODIPY 650/665, BODIPY-FL, BODIPY-R6G, BODIPY-TMR, BODIPY-TRX, Cascade Blue, Cy2, Cy3, Cy5, Cy5.5, Cy7, Cy7.5, Dylight dyes (Dylight405, Dylight488, Dylight549, Dylight550, Dylight 649, Dylight680, Dylight750, Dylight800), 6-FAM, fluorescein, FITC, HEX, 6-JOE, Oregon Green 488, Oregon Green 500, Oregon Green 514, Pacific Blue, REG, Rhodamine Green, Rhodamine Red, ROX, R-Phycoerythrin (R-PE), Starbright Blue Dyes (e.g., Starbright Blue 520, Starbright Blue 700), TAMRA, TET, Tetramethylrhodamine, Texas Red, and TRITC.

The amplified target sequence can also be sequenced using techniques known in the art, for example, nanopore sequencing (Oxford Nanopore Technologies™), reversible dye-terminator sequencing (Illumina™) and Single Molecule Real-Time (SMRT) sequencing (PacBio™). Various sequencing instruments can be used for sequencing, such as using portable Nanopore Minion™ or benchtop machines, Nanopore Promethion™, PacBio Sequel™ or Illumina HiSeq™ NextSeq™, MiSeq™, and NovaSeq™. The sequencing step can also be used for multiplex detection of several targets and/or polymorphism detection. Preferably, the sequencing of the amplified target sequence is performed on a high-throughput sequencer, such as an Illumina, PacBio or Nanopore device.

A person of ordinary skill in the art can recognize that, depending upon specific aspects of an assay, such as the technology used for sequencing the target sequence or the length of the target sequence, one may not need to introduce all of the sequences described above during the amplification step. Moreover, the tagging primer pair can be designed where one or both of the sequencing primer binding sequences are absent. For example, only one of the sequencing primer binding sequences may be sufficient for sequencing purposes if the target nucleic acid sequence is short, for example, less than about 500 bp, or a single sequencing primer is required for sequencing (e.g. PacBio). In some cases, the first or second target specific oligonucleotides can already contain at least a portion of the sequences required for sequencing. Any additional sequences that can facilitate sequencing of the double-stranded DNA containing the target nucleic acid sequence can also be introduced via one or both primers of the tagging primer pair.

The aspects described above of capturing a target nucleic acid sequence, for example, designing the target specific oligonucleotide primers and tagging primers, the length of the target nucleic acid sequences, and the first and second primer binding sequences are also applicable to the instant methods of capturing a plurality of target nucleic acid sequences.

In certain embodiments, the methods disclosed herein comprise amplifying the plurality of target nucleic acid sequences in a PCR using a tagging primer pair to produce a plurality of double-stranded tagged target sequences further comprising one or more of: first adaptor sequence, first target sequence, a target nucleic acid sequence, second target sequence, and second adaptor sequence.

In certain embodiments, multiple target sequences are captured and optionally, further analyzed, such as detected or sequenced. For a plurality of target nucleic acid sequences, a plurality of pairs of target specific oligonucleotide primers are used. Each pair of target specific oligonucleotides primers contains unique first and second target binding sequences, depending on the sequence flanking the target nucleic acid sequence. However, each of the plurality of pairs of target specific oligonucleotide primers can have the same first adaptor sequences and the same second adaptor sequences.

Accordingly, certain embodiments of the materials and methods disclosed herein provide for capturing a plurality of target nucleic acids sequences. The methods comprise the steps of:
a) annealing a plurality of first target specific oligonucleotide primers to a plurality of first target sequences, wherein each first target sequence flanks one target sequence from the plurality of target sequences, and wherein:
i) each first target specific oligonucleotide primer comprises toward the 3′ end a first target binding sequence and toward the 5′ end a first adaptor sequence;
b) amplifying the plurality of target nucleic acid sequences by extending the 3′ end of each first target specific oligonucleotide;
c) adding a plurality of second target specific oligonucleotide primers, a plurality of first tagging primers, and a plurality of second tagging primers to a plurality of amplified target sequences, wherein:
i) each second target specific oligonucleotide primer comprises toward the 3′ end a second target binding sequence and toward the 5′ end a second adaptor sequence;
ii) the plurality of first tagging primers anneals to a complement of the first adaptor sequence and the second tagging primer anneals to a complement of the second adaptor sequence; and
d) amplifying the complementary sequence to the amplified target nucleic acid sequence by extending the 3′ end of each second target specific oligonucleotide primer and amplifying the target sequence and complement thereof using the first and second tagging primers, yielding a library of tagged target sequences.

In certain embodiments, the amplification of step b) is achieved through multiple cycles of annealing/extension and denaturation.

In certain embodiments, one or both primers of the tagging or target specific oligonucleotide primer pair comprises additional sequences that can facilitate downstream sequencing of the double-stranded target nucleic acid sequences produced at the end of the final amplification step. The additional sequences that can facilitate sequencing can contain, for example, at least a portion of the sequences required for flow-cell binding and sequencing primer binding to initiate sequencing on Illumina™ platform, such as paired-end or single-read sequencing, at least a portion of the hair-pin adapter required for hairpin adapter based sequencing, such as PacBio sequencing, or at least a portion of the sequences required for properly guiding the molecules through a nanopore technology based sequencer. When the resulting molecule contains only a portion of the sequences required for sequencing, the remainder can be introduced by any other fashion know in the art, such as adapter ligation.

In certain embodiments, the plurality of target nucleic acid sequences are further analyzed, for example, detected or sequenced. The amplified target nucleic acid sequences can be detected using techniques known in the art. For example, the amplified target nucleic acid sequences can be detected based on the turbidity of the reaction, fluorescence detection or labeled molecular beacons. The aspects described above of detecting a target nucleic acid sequence are also applicable to detecting a plurality of target nucleic acid sequences.

In certain embodiments, a plurality of target nucleic acid sequences from a plurality of samples are pooled and sequenced. In such embodiments, a plurality of sequence reads is obtained corresponding to a plurality of target nucleic acid sequences from the plurality of samples. For a particular read, the unique first and/or second identifier sequences are used to allocate the read to the corresponding sample and the sequence of the captured target nucleic acid sequence in the read is compared to known databases to allocate the sequence to a target nucleic acid sequence in the sample. Thus, while only one or two sequencing primers could be used to sequence many target nucleic acid sequences in one reaction mixture, each of the sequencing reads can be systematically and accurately attributed to the appropriate source sample and appropriate target nucleic acid sequence.

In certain embodiments, a plurality of target nucleic acid sequences in a sample from a plurality of samples is amplified using a tagging primer pair that contains a unique combination of two sequence identifiers. Therefore, no two samples from the plurality of samples have the same combination of the first and the second identifiers. For example, twelve unique first identifiers and eight unique second identifiers can be used to produce ninety-six unique combinations of the first and the second identifiers. Thus, using different combinations of only twenty identifiers, ninety-six samples could be uniquely identified.

In such embodiments, for a particular read, the unique first identifier sequence and the second identifier sequence is used to allocate the read to the corresponding sample and the sequence of the captured target nucleic acid sequence in the read is compared to known databases to allocate the sequence to a target nucleic acid sequence in the sample. Thus, while only one or two sequencing primers could be used to sequence many target nucleic acid sequence in one reaction mixture, each of the sequencing reads can be systematically and accurately attributed to the appropriate source sample and appropriate target nucleic acid sequence.

Similar to detecting or sequencing a single target nucleic acid sequence, a person of ordinary skill in the art can recognize that, some of the sequences in the tagging primer pair may not be present depending upon how the tagging primer pair is designed. For example, only one identifier sequence may be present or only one sequencing primer binding sequence may be present, particularly, when the analyzed target nucleic acid sequences are short, such as less than about 500 bp, or a single sequencing primer is required for sequencing (e.g. PacBio). In some cases, the first and second target specific oligonucleotide primers can already contain at least a portion of the sequences required for sequencing, such as the sequencing primer binding sequence. Any additional sequences that can facilitate sequencing of the double-stranded DNA containing the target nucleic acid sequence can also be introduced via one or both primers of the tagging primer pair. Also, both the sequencing primer binding sequences may be absent and instead sequences can be introduced that facilitate further processing and subsequent sequencing of the double-stranded amplified target nucleic acid sequences. Such sequences include restriction enzyme sites.

Kits for carrying out the methods disclosed herein are also envisioned. Certain such kits can contain target specific oligonucleotide primers designed to capture one or more target sequences, tagging primers to amplify one or more captured target nucleic acid sequences, polymerase and other reagents for PCR, sequencing reagents, computer software program designed to process the sequencing data obtained from the assay and optionally, materials that provide instructions to perform the assay.

In certain embodiments, the kits can be customized for one or more specific target sequences. For example, a user may provide the sequences of one or more target nucleic acid sequences and a kit can be produced to carry out the assay disclosed herein for analyzing the one or more target sequences.

Reagents useful for the methods of the invention can be stored in solution or can be lyophilized. When lyophilized, some or all of the reagents can be readily stored in microwell plate wells for easy use after reconstitution. It is contemplated that any method for lyophilizing reagents known in the art would be suitable for preparing dried down reagents useful for the methods of the invention. In certain embodiments, dried down plate or reagents can comprise primers containing the barcodes used to identify a sample.

For implementation of a two-stage method in automated workflows with high sample throughput, all reagents required for each stage can be combined into a complete stage-specific mix. In certain embodiments, the complete mix of reagents can be stored frozen either in bulk format or pre-dispensed into reaction plates. For the first linear amplification stage, the complete mix of reagents can comprise of an enzyme master mix and the first adaptor-containing primer pool. For the second amplification stage, the mix of reagents can comprise of an enzyme master mix and the second adaptor-containing primer pool. In certain embodiments, the second amplification stage mix may be further combined with indexing primers by dispensing into plates containing pre-dispensed indexing primer pairs. In certain embodiments, the plates containing pre-dispensed indexing primer pairs and the second stage amplification mix may be stored frozen and may serve as reaction plates upon thawing of the first stage plates followed by addition of a sample or upon thawing of second stage plates followed by transfer of products from the first stage into the second stage plates.

In certain embodiments, pre-mixed reagents dispensed into reaction plates may be dried in the plate and rehydrated upon addition of a sample and/or water. In certain embodiments, the storage and rehydration of dried reagent mixes can enable storage and shipping at ambient temperatures (e.g., about 18° C. to about 25° C.).

In certain embodiments, such as, for example, with primer panels designed to amplify a small number of targets (e.g., less than about 3000 targets, less than about 2500 targets, less than about 2000 targets, less than about 1500 targets, less than about 1000 targets, less than 3000 targets, less than 2500 targets, less than 2000 targets, less than 1500 targets, or less than 1000 targets), the two-stage process can be reduced to a single reaction stage, in which the first adaptor-containing primer pool, the second adaptor-containing primer pool, the enzyme master mix, and the indexing primers are all provided in a single reaction well with template DNA while retaining functional performance nearly equivalent to that of the two-stage method. In certain embodiments, plates containing a complete mix of all reagents necessary to perform the one-stage method may also be stored in frozen or dried format.

All patents, patent applications, provisional applications, and publications referred to or cited herein are incorporated by reference in their entirety, including all figures and tables, to the extent they are not inconsistent with the explicit teachings of this specification. Following are examples which illustrate procedures for practicing the invention. These examples should not be construed as limiting. All percentages are by weight and all solvent mixture proportions are by volume unless otherwise noted.

### Example 1—Improved Performance of 2-Stage Linear/Exponential Amplification Protocol Compared to Exponential/Exponential Protocol with a 5000-Plex Panel

A panel of 5000 primer pairs flanking regions of interest in the maize genome was used to produce libraries following either a 2-stage Exponential/Exponential protocol (Ex/Ex), or a 2-stage Linear/Exponential protocol (Li/Ex). Each primer pair consisted of a “Forward” primer bearing a 5′ tag and a “Reverse” primer bearing a different 5′ tag.

For the Ex/Ex protocol, the first exponential reaction stage (4 replicates, 50 μL each) contained a pool of all 5000 “Forward” primers and a pool of all 5000 “Reverse” primers at 0.5 μM each, for a combined total primer concentration of 1 μM. Purified genomic DNA (20 ng) from reference strain B73 was included as template, and an amplification master mix containing RapiDxFire Hot Start Taq DNA Polymerase was included. Following amplification for 24 cycles, 10 μL of each Ex/Ex first-stage reaction was transferred directly to a new Stage 2 reaction mix (40 μL) containing a pair of indexing primers and additional amplification master mix. The 50 μL Stage 2 reactions contained indexing primers at 1 μM each. A total of 24 cycles of amplification was carried out for Stage 2.

For the Li/Ex protocol, the first Linear amplification stage (4 replicates, 10 μL each) contained the pool of 5000 “Forward” (Read 1) primers at a combined concentration of 1 μM. Purified genomic DNA (25 ng) from reference strain B73 was included as template, and the same amplification master mix was used as for the Ex/Ex protocol. Following amplification for 24 cycles, 40 μL of a Stage 2 reaction mix containing the pool of 5000 “reverse” (Read 2) primers, a pair of indexing primers, and additional amplification master mix was added to each first-stage reaction. The 50 μL Stage 2 reactions contained indexing primers at 1 μM each, and the pool of 5000 “Reverse” primers at a combined concentration of 1 μM. A total of 24 cycles of amplification was carried out for Stage 2.

For both the Ex/Ex and Li/Ex protocols, the cycling conditions for amplification were as follows:

### Stage 1

1×(95° C. for 2 min)
24×(95° C. for 15 sec; 64° C. for 1 min)
Hold at 4° C.

### Stage 2

1×(95° C. for 2 min)
6×(95° C. for 15 sec; 64° C. for 1 min)
18×(95° C. for 15 sec; 67° C. for 1 min)
Hold at 4° C.

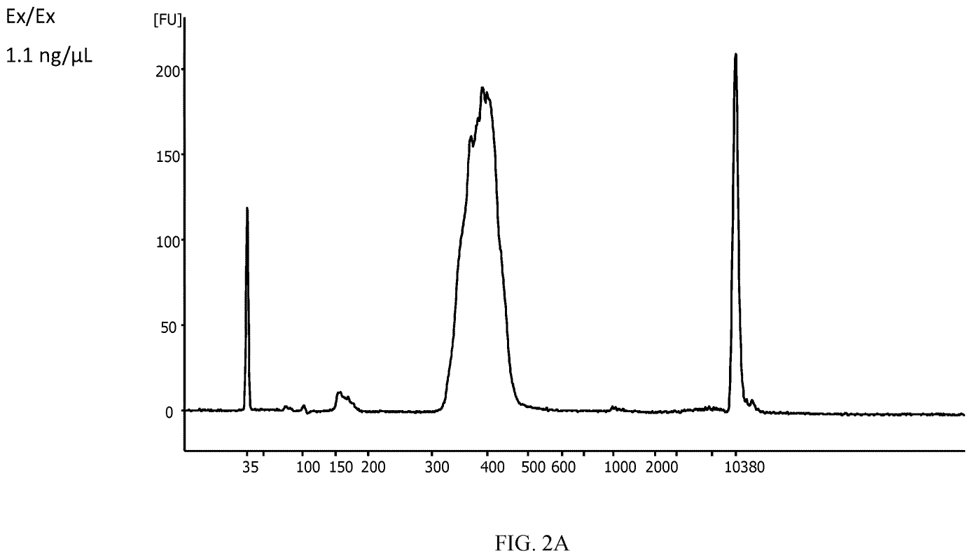

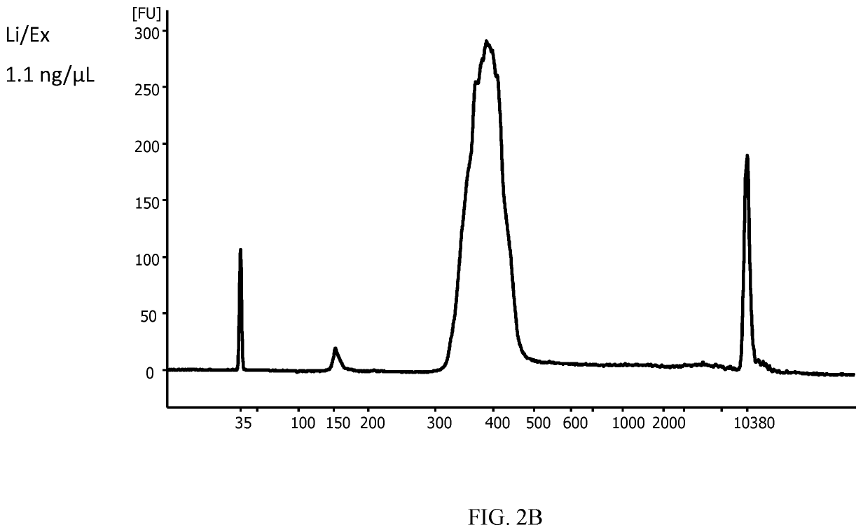

Following the completion of the Stage 2 reactions, the 4 replicates of Ex/Ex reactions and the 4 replicates of Li/Ex reactions were pooled separately, and each pool was purified using AMPure XP beads to remove unreacted primers and small products. The bead-purified library pools were quantitated using Qubit High Sensitivity DNA Assay, and samples were fractionated on a Bioanalyzer to evaluate library profiles (FIGS. 2A-2B). The 2 methods produced comparable yields of library products with similar profiles.

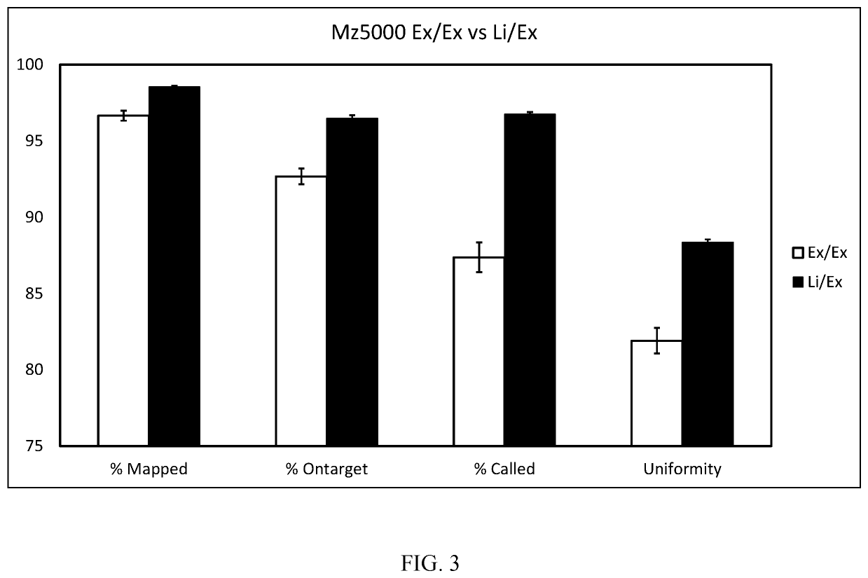

The two pools of libraries were combined for sequencing on Illumina MiSeq platform. The sequencing results are presented in FIG. 3 and Table 1. From a similar average number of total reads (923,000 reads for Ex/Ex, 889,000 reads for Li/Ex), the Li/Ex method provided sequence coverage enabling genotype calls on a significantly higher fraction of the 5000 target loci than did the Ex/Ex method: 96.8% vs. 87.4%. This improved performance of the Li/Ex method is also reflected in the average percentage of reads mapped to the genome (98.5% vs. 96.4%), percentage of reads mapped Ontarget loci (96.5% vs. 92.7%), and evenness, or “Uniformity” of coverage (defined as percentage of targets covered at ≥20% of the mean coverage depth for all 5000 targets): 88.4% vs 81.9%.

TABLE 1
Maize 5000 Sequencing Performance Metrics

| | | Seq | | % | % | |
|---|---|---|---|---|---|---|
| | Tot reads | Depth | % Mapped | Ontarget | Called | Uniformity |
| Ex/Ex | 923450 | 185 | 96.65 ± 0.33 | 92.67 ± 0.51 | 87.37 ± 0.98 | 81.91 ± 0.84 |
| Li/Ex | 889340 | 178 | 98.54 ± 0.08 | 96.47 ± 0.20 | 96.75 ± 0.14 | 88.36 ± 0.19 |

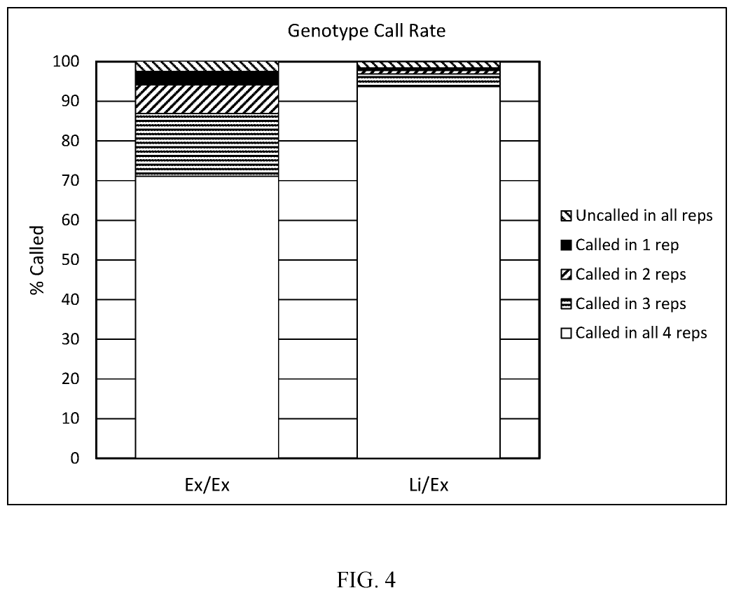

FIG. 4 and Table 2 present a comparison of the proportion of targets called consistently in all 4 replicates, or only in 1, 2, or 3 of the 4 replicates. While the Li/Ex method called 4687 of the 5000 targets (93.7%) in all 4 replicates, the Ex/Ex method called only 3554 (71.1%) in all 4 replicates. A further 791 targets were called in only 3 of the 4 Ex/Ex replicates, 358 targets in only 2 of 4 replicates, and 168 targets in only one replicate. No call was made for 129 targets in any of the 4 Ex/Ex replicates, vs. 79 uncalled targets in all 4 replicates of the Li/Ex method.

TABLE 2
Number of Targets Called

| | Ex/Ex | Li/Ex |
|---|---|---|
| 4/4 Reps | 3554 | 4687 |
| 3/4 Reps | 791 | 162 |
| 2/4 Reps | 358 | 43 |
| 1/4 Reps | 168 | 29 |
| 0/4 Reps | 129 | 79 |

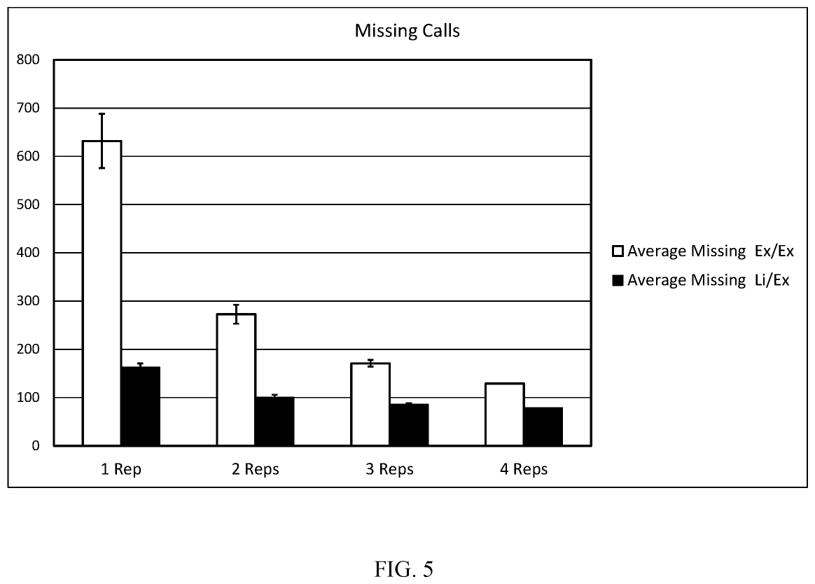

FIG. 5 and Table 3 further illustrate the inconsistency in uncalled targets among replicates of the Ex/Ex method. Individual Ex/Ex replicates failed to produce calls for 632 of 5000 targets (12.6%) on average, but any combination of 2 replicates failed to call an average of 273 targets, and any combination of 3 replicates failed to call an average of 171 targets. In contrast, each single replicate of the Li/Ex method failed to call only 163 targets on average, exceeding the performance of 3 combined replicates of the Ex/Ex method. Combining 2 or 3 replicates of the Li/Ex method only resulted in slight increases in the number of targets that could be called.

TABLE 3
Number of Missing targets in combined replicates

| | 1 Rep | 2 Reps | 3 Reps | 4 Reps |
|---|---|---|---|---|
| Ex/Ex | 632 ± 56 | 273 ± 20 | 171 ± 7 | 129 |
| Li/Ex | 163 ± 8 | 101 ± 5 | 86 ± 2 | 79 |

In summary, the 2-stage Linear/Exponential method for creating multiplex libraries for targeted genotyping by sequencing produces libraries with superior sequencing performance metrics in comparison to a standard Exponential/Exponential method. The method provides not only for high genotype call rates and high uniformity of coverage of targets within a sample, but also for consistency of target coverage across multiple samples. These properties enable the extraction of informative and consistent genotyping information.

### Example 2—Removal of Off-Target and Primer-Dimer Products by Restriction Enzyme Treatment Following First Stage Linear Amplification

The intended products of primer extension following the first linear amplification stage of the Linear/Exponential method are single-stranded. However, in some cases the targeting regions of different members of a primer pool may anneal to each other in a manner that allows one or both primers to be extended using the other primer as a template, creating double-stranded “primer-dimer” products. Further, primers within a pool may anneal to off-target sequences within extension products of other primers, again leading to generation of double-stranded products. Both primer-dimer and off-target products may be amplified exponentially during subsequent amplification cycles. Such exponential amplification can negatively impact performance of the library by degrading the uniformity of coverage depth across targets. In extreme cases, exponential amplification of unwanted double-stranded byproducts in the first stage may overtake the reaction, rendering the final library useless for genotyping.

The inclusion of a sequence comprising one strand of a recognition site for a double-strand specific restriction enzyme within the Adaptor 1 portion of the “Forward” primer pool provides an opportunity for specific removal of primer-dimer and off-target products. Following the completion of the first stage linear amplification, a restriction enzyme may be added to the reaction products to digest these unwanted double-stranded products. The restriction enzyme may be combined with the components of the second stage reaction, and the digestion is carried out at a temperature permissive for the restriction enzyme but restrictive for amplification by the DNA polymerase. The restriction enzyme may then be heat-inactivated before the second-stage exponential amplification reaction is initiated. Cleavage of the undesired double-stranded products of the first linear amplification stage within the universal tag region prevents the further amplification of these products by indexing primers during the second exponential amplification stage. The desired double-stranded products of the second exponential amplification stage, initiated by priming of the “Reverse” primer pool on the extension products of the first stage, will be unaffected by the inactivated restriction enzyme.

In the example presented here, type IIS restriction enzyme BspQI was used to digest double-stranded products following the Stage 1 linear amplification, taking advantage of the occurrence of a BspQI recognition site within the Adaptor 1 portion of the “Forward” primer pool.

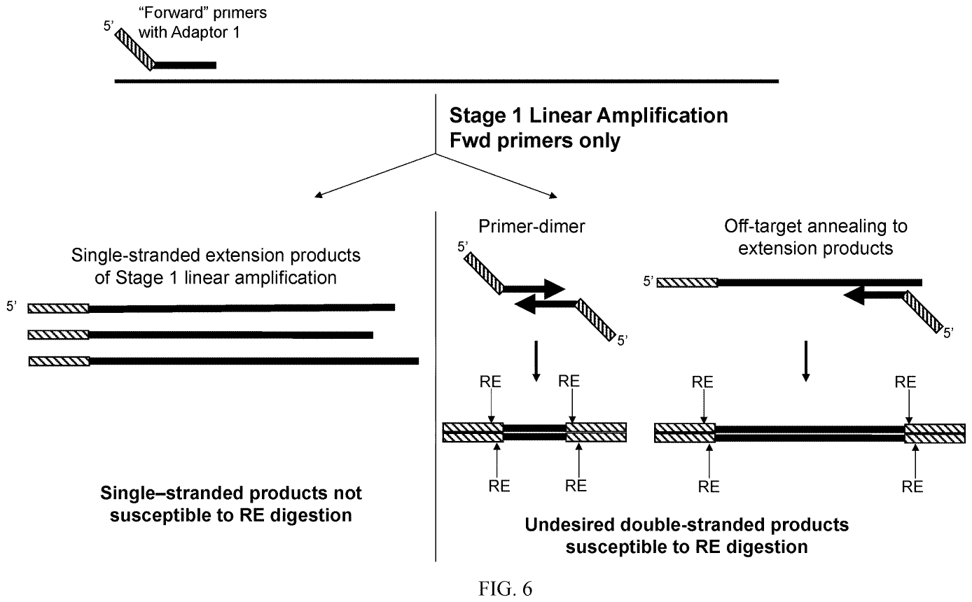

FIG. 6 presents a schematic illustration of anticipated products of a first linear amplification reaction performed with only Adaptor 1-containing Forward primers, including the intended single-stranded extension products and some potential double-stranded products arising from primer-dimer interactions or off-target priming.

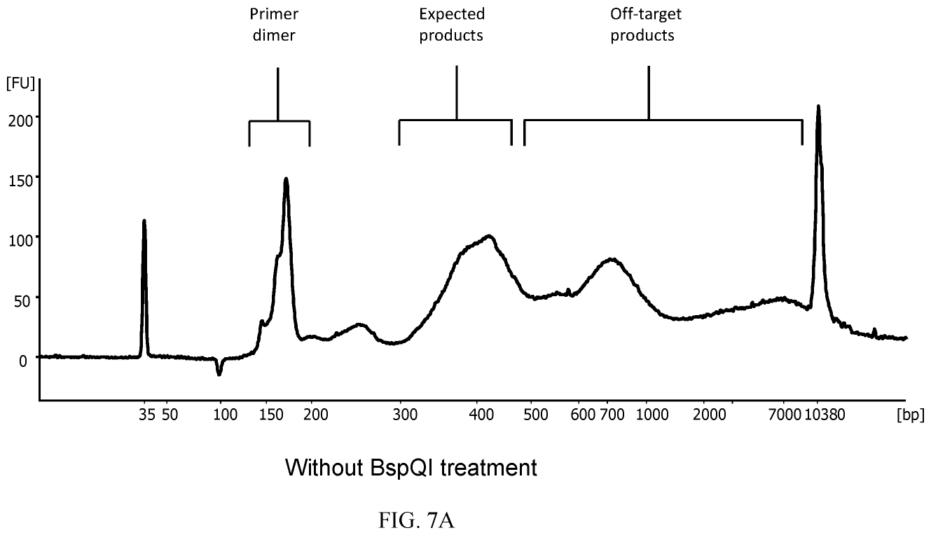

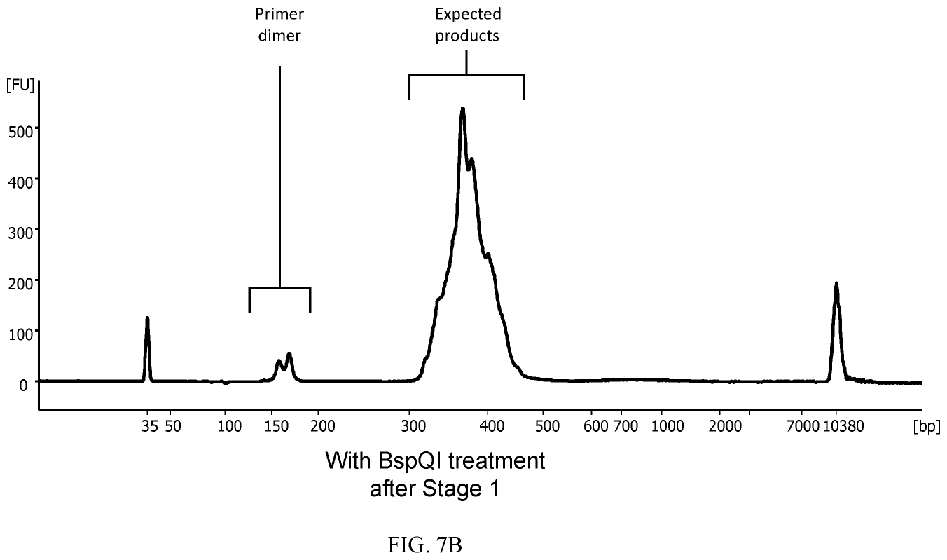

FIG. 7A-7B shows bioanalyzer traces of libraries produced with a panel of 960 primer pairs targeting regions of interest within the soy genome. FIG. 7A is an example of products of library preparation following the Linear/Exponential protocol, in which products of the first linear amplification reaction performed with Forward primers only were utilized directly in a second exponential amplification with Reverse primers and indexing primers without restriction enzyme treatment. Products include a major peak of primer-dimer sized products as well as a broad distribution of products of apparent sizes up to 10 kb. A minority of products are consistent with expected library fragment sizes. FIG. 7B shows products from the same primer pools and protocol, except that Stage 1 products were treated with restriction enzyme BspQI (New England Biolabs) before initiation of Stage 2 cycling. The major products are library fragments of the expected size (~300-450 bp) and a small amount of primer-dimer sized products (150-170 bp).

For the untreated libraries, the first stage linear amplification reaction (10 μL) contained the “Forward” Adaptor 1-containing primer pool at a total concentration of 1 μM, 10 ng of purified Soy genomic DNA (BioChain Institute, Inc.), and amplification master mix containing RapiDxFire Hot Start Taq DNA Polymerase. After 30 cycles of amplification, 40 μL of a Stage 2 reaction mix containing the pool of 960 “Reverse” Adaptor 2-containing primers, a pair of indexing primers, and additional amplification master mix was added to the first-stage reaction. The Stage 2 reactions (50 μL total) contained indexing primers at 1 μM each, and the pool of 960 “Reverse” primers at a combined concentration of 1 μM. A total of 25 cycles of amplification was carried out for Stage 2. Cycling conditions were as follows:

### Stage 1

1×(95° C. for 2 min)
30×(95° C. for 15 sec; 64° C. for 1 min)
Hold at 4° C.

### Stage 2

1×(95° C. for 2 min)
8×(95° C. for 15 sec; 64° C. for 1 min)
17×(95° C. for 15 sec; 67° C. for 1 min)
Hold at 4° C.

For the BspQI-treated libraries, the reactions and cycling conditions were the same except that the 40 μL Stage 2 reaction mix also contained 10 units of BspQI, and the 50 μL assembled Stage 2 reactions were incubated at 45° C. for 20′, followed by 80° C. for 20′ to inactivate BspQI before the final 25 cycles of amplification were initiated.

Table 4 presents some metrics from sequence analysis of the BspQI-treated libraries. The untreated libraries were not sequenced. The results show that treatment with BspQI enables generation of libraries with excellent performance characteristics (91.4% genotype call rate, 80.3% Uniformity of target coverage depth) with a panel that otherwise produced poor libraries with a high proportion of primer-dimer and off-target products.

TABLE 4
Sequencing Performance Metrics for Soy 960 panel with
BspQI treatment. Values are averages from 2 replicates.

| | % Mapped | % Ontarget | % Called | Uniformity |
|---|---|---|---|---|
| Soy 960 + BspQI | 91.5 | 86.0 | 91.4 | 80.3 |
| (Ave.) | | | | |

### Example 3—Crude Extract Compatibility of the 2-Stage Linear/Exponential Amplification Method

The source and quality of DNA samples are important considerations for genotyping workflows. While some genotyping technologies may require highly purified DNA, the ability to use crude extracts is highly desirable when high sample throughput is required. Extraction methods based on the “HotSHOT” procedure (Truett et al., 2000) have become widely favored for preparation of crude extracts from agricultural samples, including plant leaf and seed tissue.

HotSHOT extracts prepared from Maize leaf punch samples (reference strain B73) were tested for compatibility with the Linear/Exponential method. For each extraction, 2 dried leaf punches (6 mm diameter each) were ground to a powder in plastic tubes using a Geno grinder tissue homogenizer at 1750 rpm for 2′ with a 4 mm metal bead. To the ground tissue, 200 μL of 25 mM NaOH were added. Samples were incubated at 60° C. for 60 min, cooled to room temperature, and centrifuged for 10 min at 2400×g. The cleared supernatant was transferred to a clean 1.5 mL tube. Extracts were added directly to the first linear amplification reaction stage of Linear/Exponential library reactions without further treatment, or after neutralization with an equal volume of 40 mM Tris-HCl with a pH of 5, or a dilution with an equal volume of H₂O.

Libraries were prepared following the 2-stage Linear/Exponential protocol with a panel of 1152 primer pairs flanking regions of interest in the maize genome. The first Linear amplification reaction stage (10 μL total) contained the pool of 1152 “Forward” primers at a combined concentration of 1 μM. 2 μL of undiluted crude extract, or 4 μL of extract that had been diluted with Tris-HCl or H₂O were included, and the amplification was performed with a master mix containing RapiDxFire Hot Start Taq DNA Polymerase. Following 24 cycles of linear amplification, 40 μL of a Stage 2 reaction mix containing the pool of 1152 “Reverse” primers, a pair of indexing primers, and additional amplification master mix was added to each first-stage reaction. The 50 μL Stage 2 reactions contained indexing primers at 1 μM each, and the pool of 1152 “Reverse” primers at a combined concentration of 1 μM. A total of 24 cycles of amplification was carried out for Stage 2. Following Stage 2, products were purified with Ampure XP beads to remove unreacted primers and small products. Library fragment size distribution was analyzed on a Bioanalyzer, and libraries were sequenced on Illumina MiSeq.

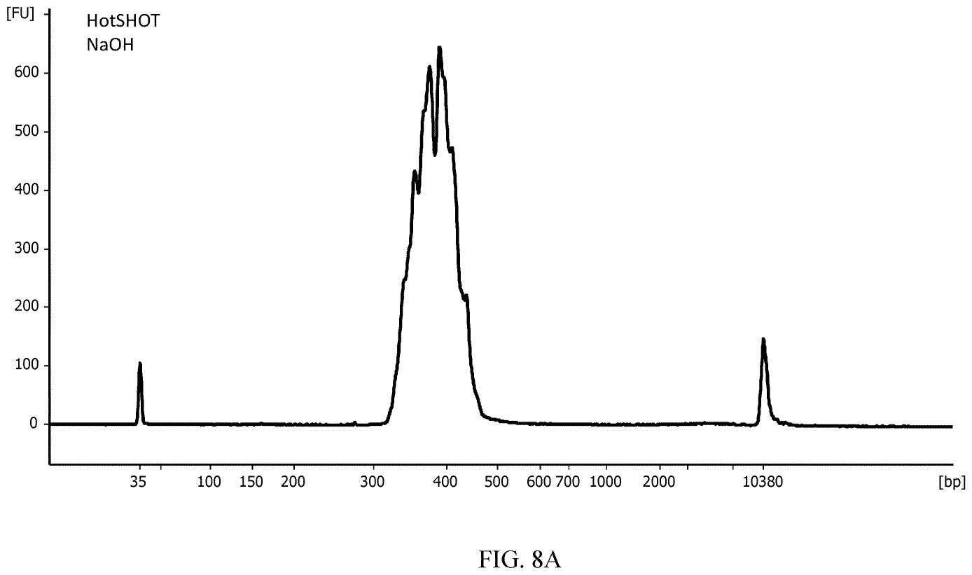

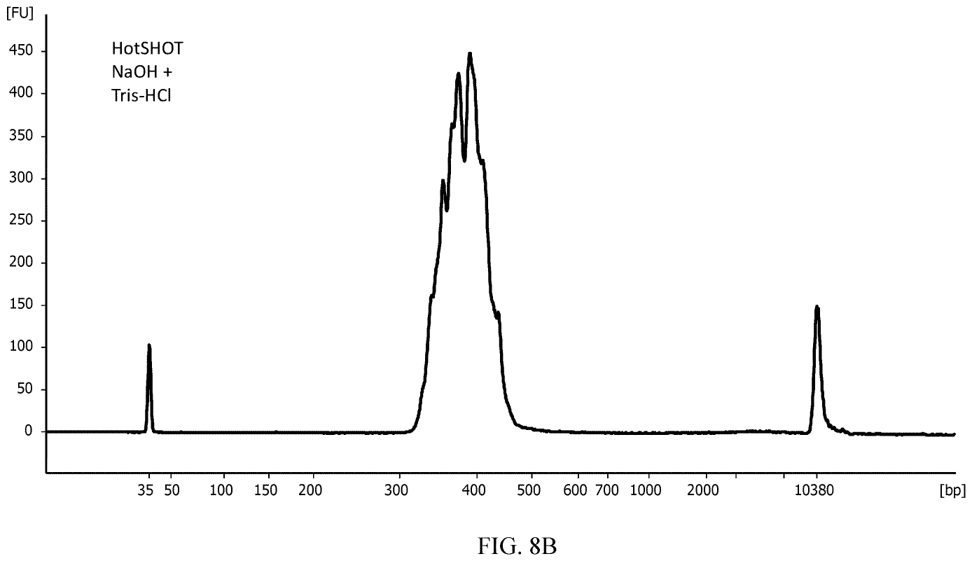

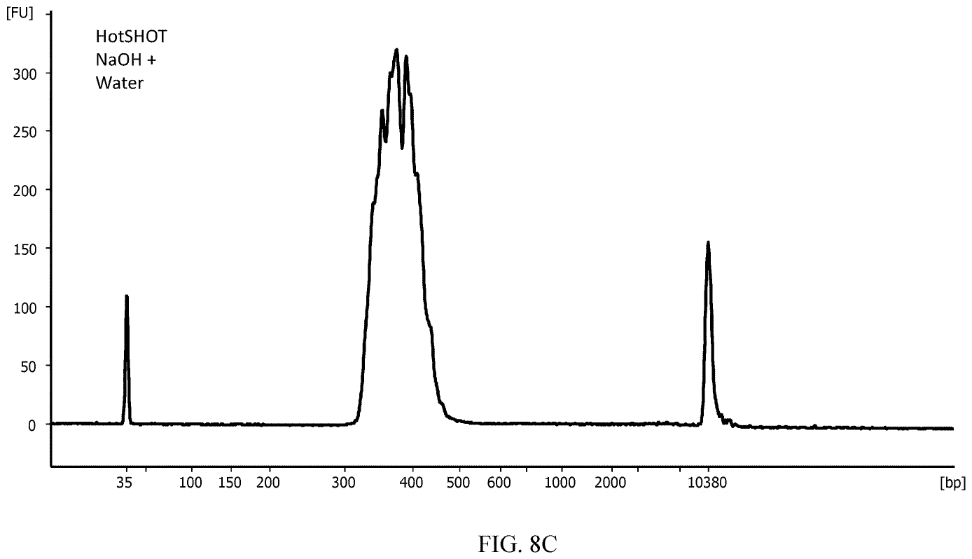

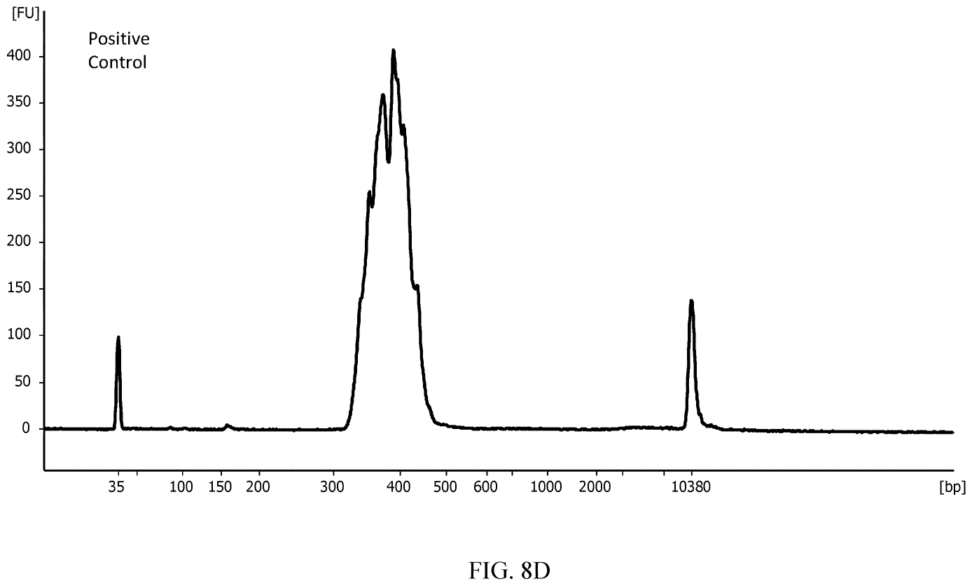

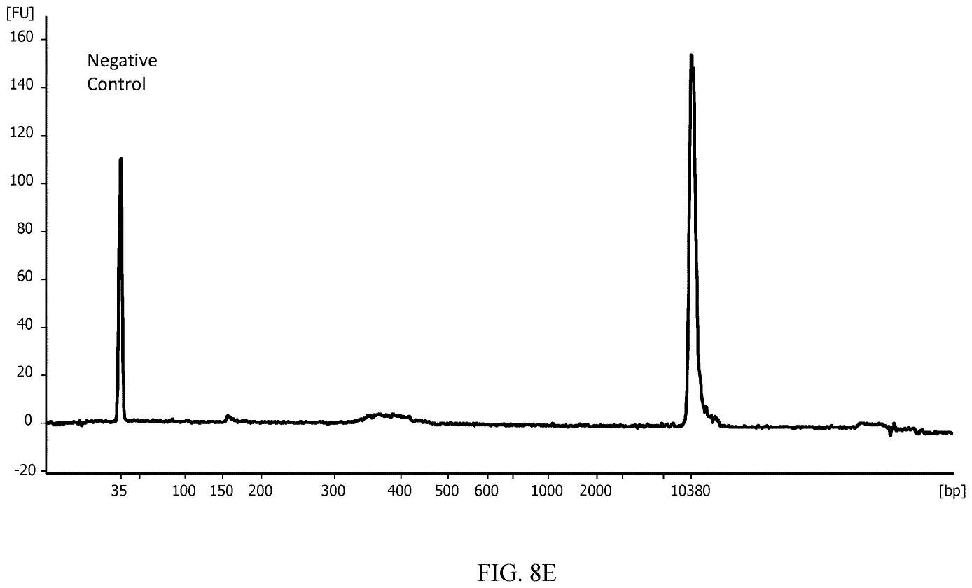

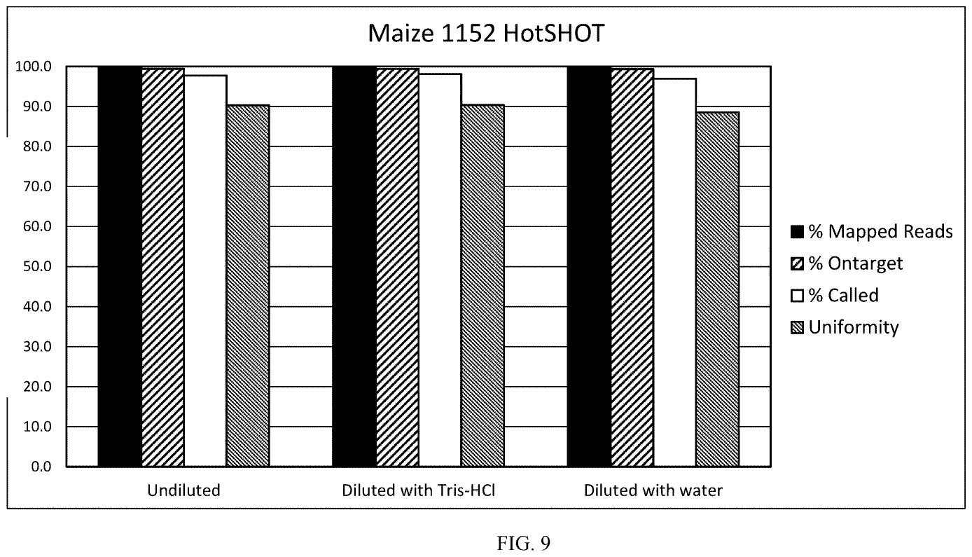

FIGS. 8A-8E show bioanalyzer traces for libraries prepared from HotSHOT extracts without dilution, or from extracts that had been diluted with an equal volume of either 40 mM Tris-HCl at a pH of 5.0 or water. Control libraries were produced with purified Maize B73 DNA (10 ng) or no DNA. FIG. 9 and Table 5 present key metrics from sequence analysis. The results show that high-quality libraries were produced from HotSHOT crude extract samples with the Linear/Exponential method, with >99% of reads mapped to target loci for all 3 conditions. Genotype calls were made for 97% to 98% of target loci at an average sequencing depth of 139 reads per target, and very high uniformity of target coverage (88-90%) was achieved.

TABLE 5
Sequencing performance metrics for Maize 1152 panel with HotSHOT crude extract.

| | Tot | Ave Seq | % Mapped | % | % | |
|---|---|---|---|---|---|---|
| | Reads | Depth | Reads | Ontarget | Called | Uniformity |
| Undiluted | 160000 | 139 | 99.68 | 99.42 | 97.74 | 90.28 |
| Diluted with Tris | 160000 | 139 | 99.65 | 99.40 | 98.09 | 90.36 |
| Diluted with water | 160000 | 139 | 99.64 | 99.34 | 96.96 | 88.54 |

All patents, patent applications, provisional applications, and publications referred to or cited herein are incorporated by reference in their entirety, including all figures and tables, to the extent they are not inconsistent with the explicit teachings of this specification.

It should be understood that the embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

### REFERENCES

1 Campbell, N. R., Harmon, S. A., and Narum, S. R. (2015). Genotyping-in-Thousands by sequencing (GT-seq): A cost effective SNP genotyping method based on custom amplicon sequencing. Mol. Ecol. Resour. 15, 855-867. doi: 10.1111/1755-0998.12357.
2. Gnirke, A., Melnikov, A., Maguire, J., Rogov, P., LeProust, E. M., Brockman, W., et al. (2009). Solution hybrid selection with ultra-long oligonucleotides for massively parallel targeted sequencing. Nat Biotechnol 27, 182-189. doi:nbt.1523 [pii]10.1038/nbt. 1523.
3. Shen, P., Wang, W., Krishnakumar, S., Palm, C., Chi, A.-K., Enns, G. M., et al. (2011). High-quality DNA sequence capture of 524 disease candidate genes. Proc. Natl. Acad. Sci. U.S.A 108, 6549-54. doi: 10.1073/pnas.1018981108.
4. Truett, G. E., Heeger, P., Mynatt, R. L., Truett, A. A., Walker, J. A. and Warman, M. L. (2000). Preparation of PCR-Quality Mouse Genomic DNA with Hot Sodium Hydroxide and Tris (HotSHOT). BioTechniques 29, 52-54. doi: 10.2144/00291bm09.

1-29. (canceled)

30. A method for amplifying a target nucleic acid sequence, the method comprising the steps of:
a) annealing a first target specific oligonucleotide primer to the target nucleic acid sequence, wherein:
the first target specific oligonucleotide primer comprises a first target binding sequence toward a 3′ end and a first adaptor sequence toward a 5′ end;
b) amplifying the target nucleic acid sequence by extending the 3′ end of the first target specific oligonucleotide primer;
c) adding a second target specific oligonucleotide primer, a first tagging primer, and a second tagging primer to the amplified target nucleic acid sequence, wherein:
the second target specific oligonucleotide primer comprises a second target binding sequence complementary to the amplified target nucleic acid sequence toward a 3′ end and a second adaptor sequence toward a 5′ end,
the first tagging primer anneals to a complement of the first adaptor sequence and the second tagging primer anneals to a complement of the second adaptor sequence;
d) amplifying the complementary sequence to the amplified target nucleic acid sequence by extending the 3′ end of the second target specific oligonucleotide primer; and
e) amplifying the target sequence and complement thereof using the first and second tagging primers, yielding a library of tagged target sequences.

31. The method of claim 30, wherein the first adaptor sequence comprises a recognition site for a restriction enzyme.

32. The method of claim 31, further comprising treating the products of step b) with a restriction enzyme.

33. The method of claim 30, wherein the first target binding sequence comprises at least 10 bases that are complementary to the target nucleic acid sequence.

34. The method of claim 30, wherein the target nucleic acid sequence is between about 10 bp and about 100 bp, between about 100 bp and about 300 bp, or between about 300 bp and about 500 bp.

35. The method of claim 30, wherein each of the first or second target specific oligonucleotide primers contains between about 20 and about 60 nucleotides.

36. The method of claim 35, wherein the first or second target binding sequence is between about 10 and about 30 nucleotides.

37. The method of claim 30, comprising amplifying the target nucleic acid sequence or complementary sequence thereof in a polymerase chain reaction (PCR) using a DNA polymerase to produce copies of the target sequence in single-stranded form.

38. The method of claim 30, further comprising sequencing the library of tagged target sequences.

39. The method of claim 38, wherein the sequencing comprises nanopore sequencing, reversible dye-terminator sequencing, Single Molecule Real-Time (SMRT) sequencing, or paired-end sequencing.

40. A method for capturing a plurality of target nucleic acid sequences, comprising the steps of:
a) annealing a plurality of first target specific oligonucleotide primers to a plurality of first target sequences, wherein each first target sequence flanks one target nucleic acid sequence from the plurality of target nucleic acid sequences, and wherein:
i) each first target specific oligonucleotide primer comprises toward the 3′ end a first target binding sequence and toward the 5′ end a first adaptor sequence, and
b) amplifying the plurality of target nucleic acid sequences by extending the 3′ end of each first target specific oligonucleotide;
c) adding a plurality of second target specific oligonucleotide primers, a plurality of first tagging primers, and a plurality of second tagging primers to the plurality of amplified target sequences of step b), wherein:
i) each second target specific oligonucleotide primer comprises toward the 3′ end a second target binding sequence and toward the 5′ end a second adaptor sequence,
ii) the plurality of first tagging primers anneals to a complement of the first adaptor sequence and the second tagging primer anneals to a complement of the second adaptor sequence;
d) amplifying the complementary sequences to the plurality of amplified target nucleic acid sequences by extending the 3′ end of each second target specific oligonucleotide primer; and
e) amplifying the target sequence and complement thereof using the first and second tagging primers, yielding a library of tagged target sequences.

41. The method of claim 40, wherein the first adaptor sequence comprises a recognition site for a restriction enzyme.

42. The method of claim 41, further comprising treating the products of step b) with a restriction enzyme.

43. The method of claim 40, wherein each first target binding sequence comprises at least 10 bases that are complementary to each target nucleic acid sequence.

44. The method of claim 40, wherein each target nucleic acid sequence is between about 10 bp and about 100 bp, between about 100 bp and about 300 bp, or between about 300 bp and about 500 bp.

45. The method of claim 40, wherein each of the first or second target specific oligonucleotide primers contains between about 20 and about 60 nucleotides.

46. The method of claim 40, wherein each first or second target binding sequence is between about 10 and about 30 nucleotides.

47. The method of claim 40, comprising amplifying the plurality of target nucleic acid sequences or complementary sequences thereof in a polymerase chain reaction (PCR) using a DNA polymerase to produce copies of the target sequences in single-stranded form.

48. The method of claim 47, further comprising sequencing the library of tagged target sequences.

49. The method of claim 48, wherein the sequencing comprises nanopore sequencing, reversible dye-terminator sequencing, Single Molecule Real-Time (SMRT) sequencing, or paired-end sequencing.

50. A kit comprising one or more pairs of primers, each pair of primers comprising a first target specific oligonucleotide primer and a second target specific oligonucleotide primer, wherein:
i) the first target specific oligonucleotide primer comprises a first target binding sequence toward a 3′ end and a first adaptor sequence toward a 5′ end; and
ii) the second target specific oligonucleotide primer comprises a second target binding sequence complementary to the amplified target nucleic acid sequence toward a 3′ end and a second adaptor sequence toward a 5′ end.

51. The method of claim 30, wherein in the target nucleic acid sequence or the plurality of first and/or second target sequences is/are a cDNA sequence reverse transcribed from a RNA sequence.

52. The method of claim 30, wherein no purification step is performed after an amplification in steps b) or d).

53. The method of claim 30, wherein steps of annealing and amplifying the target sequences in steps a) and b) are repeated.
